(19) United States
(12) Patent Application Publication
(10) Pub. No.: US 2024/0217723 A1
(43) Pub. Date: Jul. 4, 2024

(54) Bandage Storage and Dispenser Device

(71) Applicant: Stanley Hergenreter, Brush, CO (US)

(72) Inventor: Stanley Hergenreter, Brush, CO (US)

(21) Appl. No.: 18/497,024

(22) Filed: Oct. 30, 2023

Related U.S. Application Data
(60) Provisional application No. 63/436,738, filed on Jan. 3, 2023.

(51) Int. Cl. B65D 83/08; A61F 15/00; B65D 85/672

(52) U.S. Cl. CPC B65D 83/0841; A61F 15/002; B65D 85/672; B65D 2583/085

(57) ABSTRACT

A bandage storage and dispenser device usable for dispensing individual bandages when a leading bandage is pulled by a user. The device includes a central wheel for accommodating a roll of sequentially or continuously connected bandages. The roll is unrolled when the leading bandage is pulled by a user and detached from the next bandage along a perforation line disposed therebetween. The perforation line is torn using a cutter positioned at a dispensing opening of the device for releasing the leading bandage. The roll can be replaced by opening a pivoting portion of the device and a spindle supports the unrolled portion of the roll. In some embodiments, a protective covering of the bandage can be removed inside the device by a plate and the covering is released from a separate opening in the device.

### CROSS-REFERENCE TO RELATED APPLICATION

The present application claims priority to, and the benefit of, U.S. Provisional Application No. 63/436,738, which was filed on Jan. 3, 2023 and is incorporated herein by reference in its entirety.

### FIELD OF THE INVENTION

The present invention relates generally to the field of bandage dispenser devices. More specifically, the present invention relates to a novel sealed bandage storage and dispenser device that stores bandages in the form of a roll and dispenses individual bandages from an opening when a bandage is pulled by a user. The individual bandages are detachable through perforations positioned between each pair of the consecutive bandages. A cutter at the opening detaches the bandages at perforations and thus releases the leading bandage. The bandage can be released with protective covering thereon or without protective covering depending on the design of the device. The device is portable and can be carried easily in a pocket. Accordingly, the present disclosure makes specific reference thereto. Nonetheless, it is to be appreciated that aspects of the present invention are also equally applicable to other like applications, devices, and methods of manufacture.

### BACKGROUND

By way of background, people generally apply bandages over a wound, a cut, a scrape, or any other injury. Bandages are one of the first forms of first aid used on a wound and prevent infection by keeping the wound clean and shielding it from external contaminants. Additionally, bandages help in controlling bleeding and provide support to the injured area during the healing process. Bandages soak up blood and other fluids that might ooze from the wound and serve as a physical barrier between the wound and the external environment.

Commonly, bandages are individually packaged in sachets. However, sachets carry only a single bandage and opening individual sachets one by one and the applying on a wound takes time and is inconvenient.

In clinics, hospitals, and homes, bandages are often stored in boxes. Retrieving bandages from a box can be frustrating due to the need to open the lid, search for the desired size or type of bandage, and then pull a bandage out from the stack. Additionally, the bandages in the box may not remain clean and sterile. Conventional bandage boxes are also a bit bulky and cannot be carried easily during travelling. People desire a more user-friendly storage and dispenser for bandages that overcomes the problems associated with conventional bandage boxes.

Therefore, there exists a long felt need in the art for an improved storage and dispenser device for bandages. There is also a long felt need in the art for a bandage dispenser device that enables users to quickly obtain a bandage therefrom. Additionally, there is a long felt need in the art for a bandage storage and dispenser device that stores the bandages in a clean and sterile manner. Moreover, there is a long felt need in the art for a bandage dispenser device that enables a user to easily pull a bandage from the device without requiring the user to open the device. Further, there is a long felt need in the art for a novel bandage dispenser device that offers a user-friendly solution for quick and efficient wound care. Furthermore, there is a long felt need in the art for a bandage dispenser that is compact, portable, and provides the convenience of a tape dispenser with the sanitary benefits of individually stored bandages. Finally, there is a long felt need in the art for a bandage storage and dispenser device that saves considerable time and effort while improving sanitary conditions when applying a bandage onto a wound.

The subject matter disclosed and claimed herein, in one embodiment thereof, comprises a portable bandage dispenser device. The device is designed to store a roll of sequentially or continuously connected bandages and to expedite the retrieval and application of bandages. The device comprises a roller which has a roll of bandages wrapped therearound, a dispensing opening configured to release an individual bandage from the roll of bandages, the dispensing opening includes a cutter, the cutter having a plurality of teeth for detaching a leading bandage from a subsequent or following bandage enabling a user to pull the leading bandage to dispense the bandage. The device can dispense a bandage with a cover thereon or without the cover.

In this manner, the bandage storage and dispensing device of the present invention accomplishes all of the forgoing objectives and provides users with a pull dispenser for individual bandages to cover scrapes, cuts, and other injuries. The device enables the user to quickly obtain a bandage and cover a wound. The device is portable, can be carried easily, keeps the bandages clean, and enables users to pull or dispense individual bandages conveniently without much effort.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a portable bandage dispenser device. The device is designed to expedite the retrieval and application of bandages. The device comprises of a wheel which has a roll of bandages wrapped therearound, a dispensing opening configured to release an individual bandage from the roll of bandages, the dispensing opening includes a cutter, the cutter having a plurality of teeth for detaching a leading bandage from a subsequent bandage enabling a user to pull the leading bandage. The device dispenses individual bandages with a protective cover covering the bandage which is easy to be peeled off for application on a wound.

In yet another embodiment, a bandage storage and dispenser device is disclosed. The device includes a housing having a dispensing opening at the front end thereof, a roller disposed inside the housing across opposing sides of the housing, the roller is configured to roll to dispense one or more bandages through the dispensing opening, the roll includes a plurality of sequentially or continuously connected bandages, wherein a leading bandage is pulled by a user to dispense from the dispensing opening. A spindle and the roller together unroll one or more bandages from the roller when a leading bandage from the dispensing opening is pulled by a user.

In another aspect of the present invention, a bandage dispenser device is disclosed. The dispenser device includes a dispensing opening for dispensing individual bandages, a second opening for dispensing protective shield or cover covering the dispensed bandage, a roller for accommodating a roll of sequentially or continuously connected bandages, a plate for removing the cover, film, or shield from the bandage, and a cutter having a plurality of cutting teeth for detaching a preceding bandage from the next bandage wherein the cutter detaches the bandages along a perforation line disposed between the bandages.

In a further embodiment of the present invention, the roll of the bandages included in the dispenser device has a plurality of serially connected bandages and a perforation line is disposed between the two consecutive bandages, wherein the bandages are detached along the perforation line.

The device of the present invention keeps the band aids clean and makes retrieving them much quicker, making the entire bandage application process much more sanitary and less cumbersome. The device saves considerable time and effort while improving sanitary conditions when applying bandages.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of bandage storage and dispensing device of the present invention in accordance with the disclosed architecture;

FIG. 2 illustrates a perspective view showing the pivotable portion in a pivoting position for enabling access of the roller in accordance with the disclosed structure;

FIG. 3 illustrates a perspective view showing a user pulling bandages from the bandage storage and dispensing device of the present invention in accordance with the disclosed architecture;

FIG. 4 illustrates a planar view of a portion of the bandage roll used in the bandage storage and dispenser device of the present invention in accordance with the disclosed architecture;

FIG. 5 illustrates a perspective view of another embodiment of the bandage assembly and dispensing device of the present invention in accordance with the disclosed structure; and

FIG. 6 illustrates an enlarged view of the bandage pulling mechanism of the bandage assembly and dispensing device of FIG. 5 in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long felt need in the art for an improved storage and dispenser device for bandages. There is also a long felt need in the art for bandage dispenser device that enables users to quickly obtain a bandage therefrom. Additionally, there is a long felt need in the art for a bandage storage and dispenser device that stores the bandages in a clean and sterile manner. Moreover, there is a long felt need in the art for a bandage dispenser device that enables a user to easily pull a bandage from the device without requiring the user to open the device. Further, there is a long felt need in the art for a novel bandage dispenser device that offers a user-friendly solution for quick and efficient wound care. Furthermore, there is a long felt need in the art for a bandage dispenser that is compact, portable, and provides the convenience of a tape dispenser with the sanitary benefits of individually stored bandages. Finally, there is a long felt need in the art for a bandage storage and dispenser device that saves considerable time and effort while improving sanitary conditions when applying on a wound.

The present invention, in one exemplary embodiment, is a bandage dispenser device. The dispenser device includes a dispensing opening for dispensing individual bandages, a second opening for dispensing protective cover, film, or shield covering the dispensed bandage, a roller for accommodating a roll of sequentially or continuously connected bandages, a plate for removing the cover from the bandage and a cutter having a plurality of cutting teeth for detaching a preceding bandage from the next bandage wherein the cutter detaches the bandages along a perforation line disposed between the bandages.

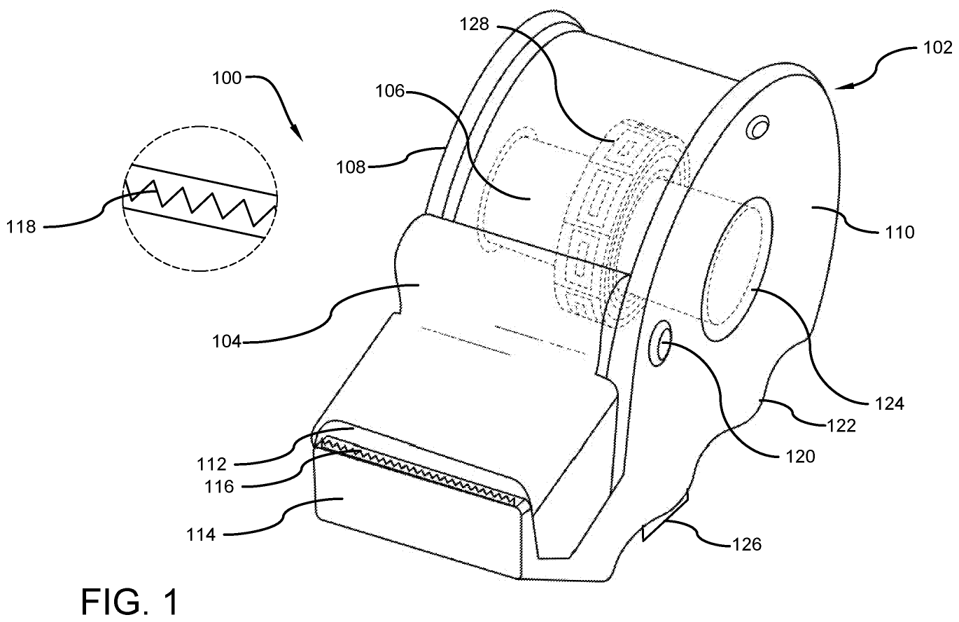

Referring initially to the drawings, FIG. 1 illustrates a perspective view of one potential embodiment of a bandage storage and dispenser device of the present invention in accordance with the disclosed architecture. The bandage storage and dispenser device 100 of the present invention is designed as a compact and portable device for storing a plurality of bandages and dispensing individual bandages therefrom. More specifically, the device 100 includes a housing 102 made of durable plastic and is configured for housing a roll of individual bandages therein. A pivotable portion 104 is configured to enable access to an interior of the housing 102 and is used for replacing the roll when all the bandages are used, thereby making the device 100 reusable. The bandage dispenser device 100 includes a roller/wheel 106 configured to roll transversely across the opposing sides 108, 110 of the device 100 for dispensing individual bandages. A plurality of bandages are arranged in the form of a roll 128 and are wrapped on the wheel 106 for dispensing individual bandages through an opening 112.

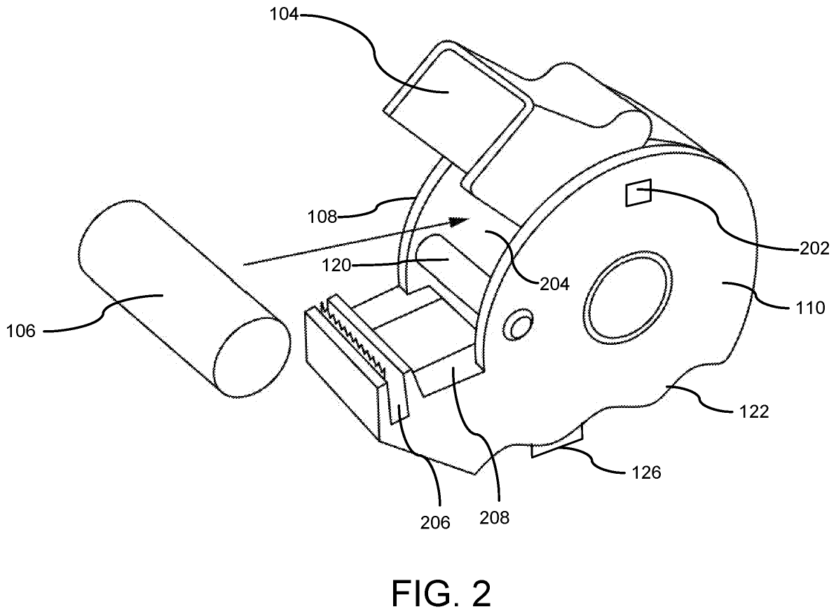

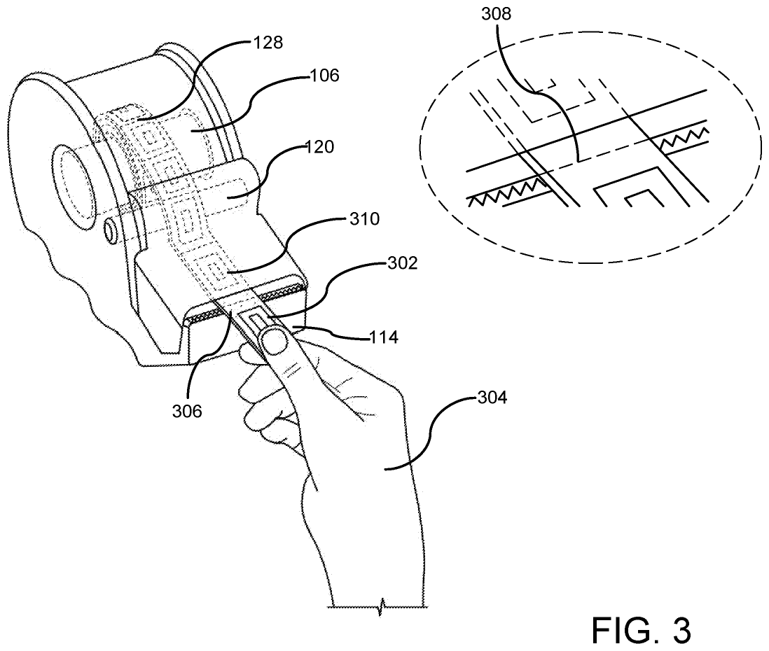

The opening 112 for dispensing individual bandages is disposed at front end 114 of the device 100 and the opening 112 includes a cutter 116 for cutting or detaching individual bandages across the perforation therebetween as illustrated in FIGS. 2 and 3. The cutter 116 includes a plurality of teeth 118 which detach serially connected bandages along the perforated lines.

A separate spindle 120 is installed inside the device 100 as illustrated in FIGS. 1 and 2 for smooth pull of the bandages from the device 100. The spindle 120 and the inside surface of the pivoting portion 104 provide a narrow passage including frictional engagement with the dispensing serially connected bandages. Bottom surface 122 of the device 100 provides an ergonomic grip when a user holds the device 100 in hand and mimics the contour of fingers. Further, the device 100 can be easily placed on any surface with the device 100 resting on the bottom surface 122. Each of the opposing sides 108, 110 have an opening 124 across which the roller 106 is supported and rotates clockwise or counterclockwise.

The device 100 is compact in design and construction and can be kept in a pocket or in a bag. Further, the bottom surface 122 can have detachable magnetic fastener 126 for enabling individuals to adhere the device 100 to any magnetic or metal surface. The bandage dispensing device 100 as per the present invention can include any suitable number of individual bandages depending upon the desired packaging, bandage sign and end use. For example, the device 100 can be configured to include at least about 10 bandages and desirably from about 10 to about 250 individual bandages, and more desirably from about 20 to about 50 individual bandages.

FIG. 2 illustrates a perspective view showing the pivotable portion in a pivoting position for enabling access of the roller 106 in accordance with the disclosed structure. The pivoting portion 104 is configured to pivot along the pivoting hinge 202 which extends across the opposing sides 108, 110. The pivoting portion 104 provides access to internal cavity 204 of the housing 102 which enables an individual to take out the roller 106 therefrom for replacing with a new roller covered with a roll of bandages. The pivoting portion 104 is secured using the receiving portions 206, 208 for a secure attachment during use of the device 100 for pulling individual bandages.

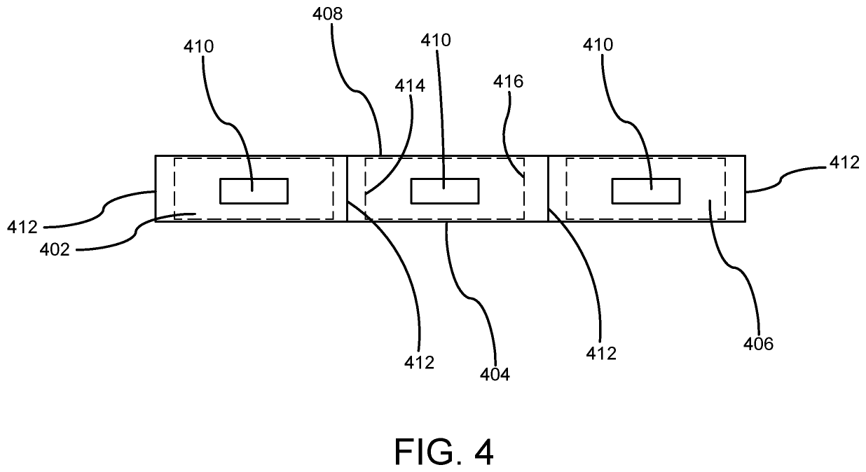

FIG. 3 illustrates a perspective view showing a user pulling bandages from the bandage storage and dispensing device of the present invention in accordance with the disclosed architecture. The individual bandages are sequentially or continuously (i.e., serially) connected to each other to form a chain of the bandages. The leading bandage 302 is pulled by a user 304 such that a protective cover 306 covering the bandage 302 (as also illustrated in FIG. 4) is torn along the perforation line 308 by the cutter 116. The perforation line 308 connects the leading bandage 302 and the subsequent bandage 310 and when the leading bandage 302 is pulled out, the subsequent bandage 310 becomes the leading bandage.

When the leading bandage 302 is pulled by the user 304, the roller 106 rolls towards the front end 114 of the device 100, thereby unrolling the roll 128 of the bandages and releasing or dispensing the individual bandages from the opening 112. The spindle 120 supports the unrolled bandages from the roller 106 and thus prevents the bandages from getting entangled and prevents the bandages from getting damaged.

FIG. 4 illustrates a planar view of a portion of the bandage roll used in the bandage storage and dispenser device of the present invention in accordance with the disclosed architecture. In the present embodiment, the bandages 402, 404, 406 are sequentially or serially connected and covered by a common protective cover 408. Each bandage is adhered to the underside of the cover 408 and the cover protects the gauze 410 of each bandage. The cover 408 includes a perforation line 412 between each pair of the bandages and while pulling a bandage as illustrated in FIG. 3, the perforation line 412 between the leading bandage 402 and the subsequent bandage 404 is torn for dispensing the leading bandage 402. A portion of the cover extends along the length of the bandage 402 and can be easily peeled off for easy and convenient use of the bandage 402.

In one embodiment of the present invention, the ends of the bandage coincide with the perforation lines 412. As an example, as illustrated, the ends 414, 416 of the bandage 404 can coincide with the perforation lines 412 positioned on both sides of the bandage 404.

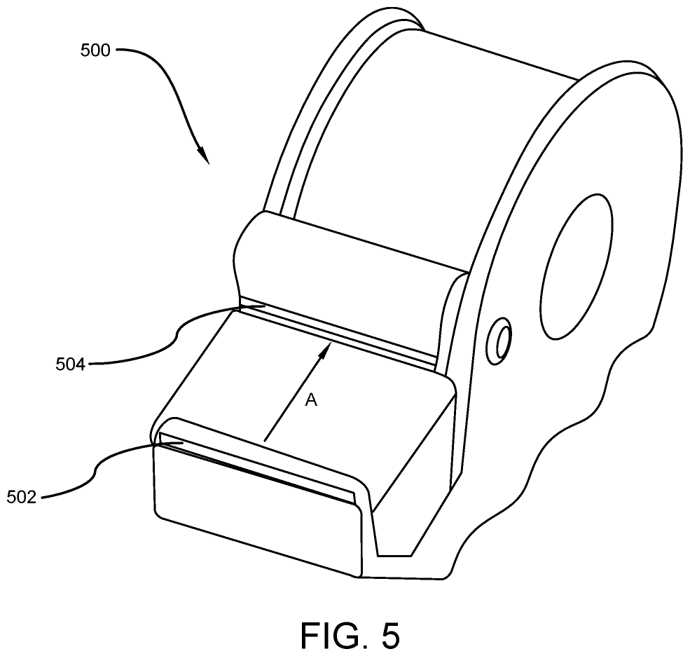

FIG. 5 illustrates a perspective view of another embodiment of the bandage assembly and dispensing device of the present invention in accordance with the disclosed structure. In the present embodiment, the device 500 enables a user to pull a bandage therefrom without the protective cover covering the bandage. A dispenser opening 502 is used for pulling a bandage and a second opening 504 is disposed which provides the cover protecting the dispensed bandage. In the present embodiment, a user is not required to peel off the protective cover.

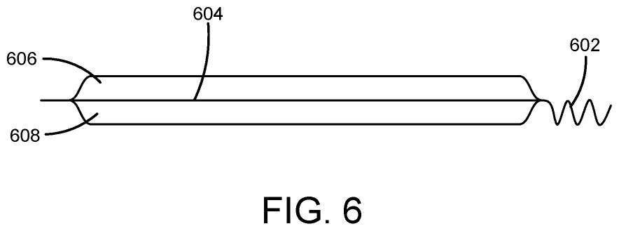

FIG. 6 illustrates an enlarged view of the bandage pulling mechanism of the bandage assembly and dispensing device 500 of FIG. 5 in accordance with the disclosed architecture. In the device 500, the cutter 602 is placed inside the device 500 and not at the dispenser opening 502. When a bandage is pulled, a cutting plate 604 removes the bandage cover 606 from the bandage 608, thereby separating the cover 606 and the bandage 608. The bandage 608 is dispensed from the opening 502 and the cover 606 is separately dispensed from the second opening 504 (as shown by arrow A in FIG. 5) of the device 500.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “bandage storage and dispenser device”, “bandage dispensing device”, “bandage assembly and dispensing device”, and “device” are interchangeable and refer to the bandage storage and dispenser device 100, 500 of the present invention.

Notwithstanding the forgoing, the bandage storage and dispenser device 100, 500 of the present invention can be of any suitable size and configuration as is known in the art without affecting the overall concept of the invention, provided that it accomplishes the above stated objectives. One of ordinary skill in the art will appreciate that the bandage storage and dispenser device 100, 500 as shown in the FIGS. 1-6 are for illustrative purposes only, and that many other sizes and shapes of the bandage storage and dispenser device 100, 500 are well within the scope of the present disclosure. Although the dimensions of the bandage storage and dispenser device 100, 500 are important design parameters for user convenience, bandage storage and dispenser device 100, 500 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A bandage dispensing device for dispensing individual bandages, the bandage dispensing device comprising:
a dispenser having a housing including an interior, a pivotal portion, a front dispensing opening, and a roller wheel for retaining a roll of bandages;
wherein said pivotable portion selectively openable for access to said interior of said housing;
wherein said housing having a first side wall and an opposing second side wall for retaining said roller wheel transversely therebetween;
wherein said roll of bandages having a continuous series of individual bandages wrapped around said roller wheel;
wherein each pair of said series of individual bandages connected by perforated lines; and
further wherein said front dispensing opening having a cutter at a terminal edge for cutting said perforated line of a leading bandage from a serially connected trailing bandage.

2. The bandage dispensing device of claim 1, wherein said cutter having a plurality of teeth for the cutting of said perforated line.

3. The bandage dispensing device of claim 2, wherein said first side wall and said second wall having a spindle therebetween, and further wherein said spindle and said pivotal portion forming a passage for frictionally retaining said serially connected bandages.

4. The bandage dispensing device of claim 3, wherein said dispenser is reusable, and further wherein a dispensed first roll of bandages is replenished with a second roll of bandages.

5. The bandage dispensing device of claim 4, wherein said housing having a finger contoured bottom surface.

6. The bandage dispensing device of claim 5, wherein said bottom surface having a magnetic bottom surface for adhering said dispenser to a metal surface.

7. The bandage dispensing device of claim 6, wherein said series of individual bandages having a count from 20 bandages to 50 bandages.

8. A bandage dispensing device for dispensing individual bandages, the bandage dispensing device comprising:
a dispenser having a housing including an interior, a pivotal portion, a first dispensing opening, a second dispensing opening, a roller wheel for retaining a roll of bandages, a cutter, and a cutting plate;
wherein said pivotable portion selectively openable for access to said interior of said housing;
wherein said housing having a first side wall and an opposing second side wall for retaining said roller wheel transversely therebetween;
wherein said roll of bandages having a continuous series of individual bandages wrapped around said roller wheel;
wherein adjacent pairs of said series of individual bandages connected by perforated lines;
wherein each of said series of individual bandages having a protective cover;
wherein said cutting plate separates said protective cover from each of said series of individual bandages when dispensing each of said series of individual bandages;
wherein said protective cover dispensed through said first dispensing opening;
wherein said cutter cutting said perforated line of a leading bandage from a serially connected trailing bandage; and
further wherein said series of individual bandages dispensed through said second dispensing opening.

9. The bandage dispensing device of claim 8, wherein said cutter having a plurality of teeth for the cutting of said perforated line.

10. The bandage dispensing device of claim 9, wherein said first side wall and said second wall having a spindle therebetween, and further wherein said spindle and said pivotal portion forming a passage for frictionally retaining said serially connected bandages.

11. The bandage dispensing device of claim 10, wherein said dispenser is reusable, and further wherein a dispensed first roll of bandages is replenished with a second roll of bandages.

12. The bandage dispensing device of claim 11, wherein said housing having a finger contoured bottom surface.

13. The bandage dispensing device of claim 12, wherein said bottom surface having a magnetic bottom surface for adhering said dispenser to a metal surface.

14. The bandage dispensing device of claim 13, wherein said series of individual bandages having a count from 20 bandages to 50 bandages.

15. A method of dispensing individual bandages, the method comprising the steps of:
providing a dispenser having a housing including an interior, a first dispensing opening, a second dispensing opening, a roller wheel, a cutter, and a cutting plate;
mounting a roll of bandages onto said roller wheel, wherein said roll of bandages having a continuous series of individual bandages including protective covers, further wherein adjacent pairs of said series of individual bandages connected by perforated lines;
retaining said roller wheel transversely between a first side wall and an opposing second side wall of said housing;
pulling said continuous series of individual bandages;
separating said protective cover from each of said series of individual bandages with said cutting plate when dispensing each of said series of individual bandages;
dispensing said protective cover through said first dispensing opening;
cutting with said cutter said perforated line of a leading bandage from a serially connected trailing bandage; and
dispensing said series of individual bandages through said second dispensing opening.

16. The method of dispensing individual bandages of claim 15, wherein said cutter having a plurality of teeth for the cutting of said perforated line.

17. The method of dispensing individual bandages of claim 16, wherein said first side wall and said second wall having a spindle therebetween, and further wherein said spindle and said pivotal portion forming a passage for frictionally retaining said serially connected bandages.

18. The method of dispensing individual bandages of claim 17, wherein said dispenser is reusable, and further wherein a dispensed first roll of bandages is replenished with a second roll of bandages.

19. The method of dispensing individual bandages of claim 18, wherein said housing having a finger contoured bottom surface.

20. The method of dispensing individual bandages of claim 19, wherein said bottom surface having a magnetic bottom surface for adhering said dispenser to a metal surface.
